(19) United States
(12) Patent Application Publication
(10) Pub. No.: US 2017/0112879 A1
(43) Pub. Date: Apr. 27, 2017

(54) METHODS OF PROTECTION AGAINST ISCHEMIA REPERFUSION INJURY

(71) Applicant: UNIVERSITY OF WASHINGTON, Seattle, WA (US)

(72) Inventors: Michael S. MULLIGAN, Seattle, WA (US); Billanna HWANG, Seattle, WA (US); Conrad W. LILES, Seattle, WA (US)

(73) Assignee: UNIVERSITY OF WASHINGTON, Seattle, WA (US)

(21) Appl. No.: 15/317,351

(22) PCT Filed: Jun. 9, 2015
(86) PCT No.: PCT/US15/34850

§ 371 Date: Dec. 8, 2016

Related U.S. Application Data
(60) Provisional application No. 62/009,529, filed on Jun. 9, 2014.

(51) Int. Cl. A61K 35/28; A61L 27/36

(52) U.S. Cl. CPC A61K 35/28; A61L 27/3683; A61L 2430/22; A61B 17/3205

(57) ABSTRACT

Provided herein are methods of treatment and prevention of ischemia reperfusion (IR) injury comprising administering a mesenchymal stem cell conditioned medium to a transplant tissue or donor.

### CROSS REFERENCE TO RELATED APPLICATIONS

This application claims benefit under 35 U.S.C. §119(e) of U.S. Provisional No. 62/009,529, filed Jun. 9, 2014 the contents of which are incorporated herein by reference in their entirety.

### FIELD OF THE INVENTION

The present disclosure is directed to methods of treatment and prevention of ischemia reperfusion (IR) injury.

### BACKGROUND

Despite advances in preservation and supportive care, ischemia reperfusion (IR) injury is a major cause of primary graft failure post-transplantation. IR injury occurs in a variety of patients (transplant, shock, cardio-pulmonary bypass). 15-30% of patients develop clinically significant reperfusion injury followed by major graft dysfunction. Patients that develop IR injury have higher incidence of acute graft rejection. For certain tissues, there are specific manifestations of IR injury, e.g., for the lung, IR injury can lead to development of early onset obliterative bronchiolitis, and chronic rejection. IR injury is a significant cause of morbidity and mortality.

### SUMMARY OF THE INVENTION

Provided herein are methods of treating, preventing, or reducing the risk of ischemia reperfusion injury in a transplant tissue. While protection from IR injury is demonstrated herein in a lung transplant model, the methods are applicable generally to protection against IR in a broad range of transplanted tissues, particularly tissues that are vascularized. As demonstrated herein, pretreatment of a subject or a transplant tissue to be transplanted with mesenchymal stem cell (MSC) conditioned medium prior to transplant induces ischemic tolerance. Consistent with these results, proinflammatory cytokines associated with ischemic injury were found to be significantly reduced, as shown herein. In addition, differentials were shown to have an increase in percentage of lymphocytes and monocytes in peripheral blood, and bronchoalveolar lavage (BAL) samples showed significant increases in AV macrophages and T cells. The data described herein indicate that protection is driven by activation and recruitment of regulatory mononuclear cells and phenotypic changes in residential cells. Data from qPCR show increases in FoxP3 expression indicate that graft infiltrating lymphocytes (GILs) are regulatory T cells. Accordingly, provided herein are novel “cell-free” approaches for inducing ischemic tolerance by pretreating a subject or a transplant tissue, prior to ischemic insult, with conditioned medium from cultured mesenchymal stem cells.

Accordingly, in some aspects, provided herein are methods of preventing, reducing the severity of, or reducing the risk of ischemia reperfusion injury to a transplant tissue comprising treating a transplant tissue with a therapeutically effective amount of an MSC (mesenchymal stem cell)-conditioned medium prior to transplanting the tissue to a transplant recipient.

In some embodiments of these methods and all such methods described herein, the treating comprises administering the MSC-conditioned medium to a transplant donor prior to excision of the transplant tissue.

In some embodiments of these methods and all such methods described herein, the MSC-conditioned medium is administered for between 15 minutes to 1 hour prior to excision of the tissue from the transplant donor.

In some embodiments of these methods and all such methods described herein, the MSC-conditioned medium is administered for at least 30 minutes prior to excision of the tissue from the transplant donor.

In some embodiments of these methods and all such methods described herein, the MSC-conditioned medium is administered to the transplant donor by a method selected from the group consisting of local administration to the tissue and systemic infusion.

In some embodiments of these methods and all such methods described herein, the MSC-conditioned medium is administered by intratracheal local administration.

In some embodiments of these methods and all such methods described herein, the treating comprises perfusing the transplant tissue with the MSC-conditioned medium ex vivo prior to transplanting the transplant tissue to the transplant recipient.

In some embodiments of these methods and all such methods described herein, the methods further comprise the step of transplanting the tissue into a transplant recipient.

In some embodiments of these methods and all such methods described herein, the transplant tissue is an organ comprising a lung or a portion or lobe thereof, a kidney, a heart, a liver or a portion thereof, a pancreas or a portion thereof, a bone, bone marrow, or a segment of bowel or other portion of the alimentary canal.

In some embodiments of these methods and all such methods described herein, the transplant tissue is a lung or a portion or lobe thereof.

In some embodiments of these methods and all such methods described herein, the MSC-conditioned medium is free of cells.

In some embodiments of these methods and all such methods described herein, the MSC-conditioned medium is serum-free.

In some embodiments of these methods and all such methods described herein, the MSC-conditioned medium is obtained from a cell culture comprising MSCs that are positive for expression of at least one of CD105, CD90, CD73, and CD106, and are negative for expression of at least one of CD45, CD34, CD14, CD19, CD11b, and HLA-DR.

In some embodiments of these methods and all such methods described herein, the MSC-conditioned medium is obtained from a cell culture comprising MSCs that are positive for expression of CD105 and are negative for expression of CD34.

Also provided herein, in some aspects, are methods for treating, preventing, or reducing the risk of ischemia reperfusion injury in a transplant tissue comprising: administering a therapeutically effective amount of an MSC (mesenchymal stem cell)-conditioned medium to a transplant donor prior to excision of a transplant tissue from the transplant donor; and excising the transplant tissue from the transplant donor.

In some embodiments of these methods and all such methods described herein, the methods further comprise the step of transplanting the excised transplant tissue into a transplant recipient.

In some embodiments of these methods and all such methods described herein, the MSC-conditioned medium is administered to the transplant donor by a method selected from the group consisting of local administration to the transplant tissue and systemic infusion.

In some embodiments of these methods and all such methods described herein, the MSC-conditioned medium is administered by intratracheal local administration.

In some embodiments of these methods and all such methods described herein, the transplant donor is administered the MSC-conditioned medium for between 15 minutes to 1 hour prior to excision of the transplant tissue from the transplant donor.

In some embodiments of these methods and all such methods described herein, the transplant donor is administered the MSC-conditioned medium for at least 30 minutes prior to excision of the transplant tissue from the transplant donor.

In some embodiments of these methods and all such methods described herein, the transplant tissue is an organ comprising a lung or a portion or lobe thereof, a kidney, a heart, a liver or a portion thereof, a pancreas or a portion thereof, a bone, bone marrow, or a segment of bowel or other portion of the alimentary canal.

In some embodiments of these methods and all such methods described herein, the transplant tissue is a lung or a portion or lobe thereof.

In some embodiments of these methods and all such methods described herein, the MSC-conditioned medium is free of cells.

In some embodiments of these methods and all such methods described herein, the MSC-conditioned medium is serum-free.

In some embodiments of these methods and all such methods described herein, the MSC-conditioned medium is obtained from a cell culture comprising MSCs that are positive for expression of at least one of CD105, CD90, CD73, and CD106, and are negative for expression of at least one of CD45, CD34, CD14, CD19, CD11b, and HLA-DR.

In some embodiments of these methods and all such methods described herein, the MSC-conditioned medium is obtained from a cell culture comprising MSCs that are positive for expression of CD105 and are negative for expression of CD34.

Provided herein, in some aspects, are methods for treating, preventing, or reducing the risk of ischemia reperfusion injury in a transplant tissue comprising: administering a therapeutically effective amount of an MSC (mesenchymal stem cell)-conditioned medium to a transplant donor prior to excision of a transplant tissue from the transplant donor; excising the transplant tissue from the transplant donor; and transplanting the excised transplant tissue into a transplant recipient.

In some embodiments of these methods and all such methods described herein, the MSC-conditioned medium is administered to the transplant donor by a method selected from the group consisting of local administration to the transplant tissue and systemic infusion.

In some embodiments of these methods and all such methods described herein, the MSC-conditioned medium is administered by intratracheal local administration.

In some embodiments of these methods and all such methods described herein, the transplant donor is administered the MSC-conditioned medium for between 15 minutes to 1 hour prior to excision of the transplant tissue from the transplant donor.

In some embodiments of these methods and all such methods described herein, the transplant donor is administered the MSC-conditioned medium for at least 30 minutes prior to excision of the transplant tissue.

In some embodiments of these methods and all such methods described herein, the transplant tissue is an organ comprising a lung or a portion or lobe thereof, a kidney, a heart, a liver or a portion thereof, a pancreas or a portion thereof, a bone, bone marrow, or a segment of bowel or other portion of the alimentary canal.

In some embodiments of these methods and all such methods described herein, the transplant tissue is a lung or a portion or lobe thereof.

In some embodiments of these methods and all such methods described herein, the MSC-conditioned medium is free of cells.

In some embodiments of these methods and all such methods described herein, the MSC-conditioned medium is serum-free.

In some embodiments of these methods and all such methods described herein, the MSC-conditioned medium is obtained from a cell culture comprising MSCs that are positive for expression of at least one of CD105, CD90, CD73, and CD106, and are negative for expression of at least one of CD45, CD34, CD14, CD19, CD11b, and HLA-DR.

In some embodiments of these methods and all such methods described herein, the MSC-conditioned medium is obtained from a cell culture comprising MSCs that are positive for expression of CD105 and are negative for expression of CD34.

Also provided herein, in some aspects, are pharmaceutical compositions comprising MSC-conditioned medium for use in administering to a tissue of a transplant donor for the treatment, prevention, or reduction of risk of ischemia reperfusion injury in a transplant tissue after transplant.

In some embodiments of these uses and all such uses described herein, the MSC-conditioned medium is administered to the transplant donor by a method selected from the group consisting of local administration to the transplant tissue and systemic infusion.

In some embodiments of these uses and all such uses described herein, the MSC-conditioned media is administered by intratracheal local administration.

In some embodiments of these uses and all such uses described herein, the transplant donor is administered the MSC-conditioned medium for between 15 minutes to 1 hour prior to excision of the transplant tissue.

In some embodiments of these uses and all such uses described herein, the transplant donor is administered the MSC-conditioned medium for at least 30 minutes prior to excision of the transplant tissue.

In some embodiments of these uses and all such uses described herein, the transplant tissue is an organ comprising a lung or a portion or lobe thereof, a kidney, a heart, a liver or a portion thereof, a pancreas or a portion thereof, a bone, bone marrow, or a segment of bowel or other portion of the alimentary canal.

In some embodiments of these uses and all such uses described herein, the transplant tissue is a lung or a portion or lobe thereof.

In some embodiments of these uses and all such uses described herein, the MSC-conditioned medium is free of cells.

In some embodiments of these uses and all such uses described herein, the MSC-conditioned medium is serum-free.

In some embodiments of these uses and all such uses described herein, the MSC-conditioned medium is obtained from a cell culture comprising MSCs that are positive for expression of at least one of CD105, CD90, CD73, and CD106, and are negative for expression of at least one of CD45, CD34, CD14, CD19, CD11b, and HLA-DR.

In some embodiments of these uses and all such uses described herein, the MSC-conditioned medium is obtained from a cell culture comprising MSCs that are positive for expression of CD105 and are negative for expression of CD34.

### DEFINITIONS

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735).

As used herein, a “mesencyhmal stem cell” refers to an adherent, immune privileged cell of non-hematopoietic origin that, at a minimum, is positive for expression of at least one of CD105, CD90, CD73, and CD106, and negative for expression of at least one of CD45, CD34, CD14, CD19, CD11b, and HLA-DR, and can undergo osteogenic, adipogenic and chondrogenic differentiation ex vivo.

As used herein, the term “MSC conditioned medium” or “mesenchymal stem cell conditioned medium” refers to cell culture medium in which MSCs (preferably human MSCs) have been cultured or maintained. MSC conditioned medium comprises soluble factors (“culture-derived growth factors”) made by the mesenchymal stem cells cultured in the medium.

The term “serum-free media,” as used herein, refers to cell culture media which is free of serum.

As used herein, “essentially cell-free” refers to a MSC conditioned medium that contains fewer than about 10%, fewer than about 5%, fewer than about 1%, fewer than about 0.1%, fewer than about 0.01%, fewer than about 0.001%, fewer than about 0.0001%, or less, than the number of cells per unit volume, as compared to the MSC culture from which it was separated.

As used herein, the terms “ischemic event,” “injury resulting from ischemia,” “injury caused by ischemia,” and “ischemic injury” refer to an injury to a cell, tissue, or organ caused by ischemia, or an insufficient supply of blood, and thus oxygen, thereby resulting in damage or dysfunction of the tissue or organ.

“Ischemia-reperfusion injury,” is a type of ischemic event that is characterized biochemically by a depletion of oxygen during an ischemic event involving interrupted blood flow followed by reoxygenation and the concomitant generation of reactive oxygen species during reperfusion.

As used herein, “transplant tissue” refers to any tissue or organ or portion thereof to be transplanted to a transplant recipient including, but not limited to, a lung or a portion or lobe thereof, a kidney, a heart, a liver or a portion thereof, a pancreas or a portion thereof, a bone, bone marrow, or a segment of bowel or other portion of the alimentary canal.

As used herein, a “transplant donor” is a subject from whom a transplant tissue or organ is obtained prior to transplantation of the tissue to a transplant recipient.

As used herein, “excision” or “removal,” as applied to a transplant tissue or organ, refers to the physical removal of the tissue or organ or portion thereof from the transplant donor. Such removal includes any surgical or non-surgical procedure by which the transplant tissue is removed and includes conventional surgical procedures, as well as laparoscopic interventions.

As used herein, a “transplant recipient” is a subject into whom a transplant tissue or organ is placed after excision or removal of the tissue from the transplant donor.

The phrase “preventing, reducing the severity of, or reducing the risk of ischemia reperfusion injury to a transplant tissue” (or variations thereof) generally refers to decreasing or reducing the degree of injury to a transplant tissue or organ caused by a depletion of oxygen during an ischemic event followed by reoxygenation and the concomitant generation of reactive oxygen species during reperfusion.

As used herein, in regard to any of the compositions, methods, and uses described herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The term “effective amount” as used herein refers to the amount of MSC-conditioned medium needed to alleviate at least one or more symptom or marker of ischemic reperfusion injury, and relates to a sufficient amount of the MSC-conditioned medium required to provide the desired effect.

As used herein, the terms “administering,” “delivering,” and “introducing” are used interchangeably and refer to the placement of MSC conditioned medium described herein into a transplant donor or to a transplant tissue by a method or route which eventually results in at least partial localization of such MSCconditioned medium at a desired site, such that a desired effect(s) is produced.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All patents and other publications identified herein, both supra and infra, are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### BRIEF DESCRIPTION OF THE DRAWINGS

FIG. 1 shows the treatment schema for the eleven (11) cohorts studied in Table 1.

FIG. 2 shows lung permeability for MSC clones O and H. Administration of conditioned media from either clone O or H prior to ischemia reperfusion injury confers protection.

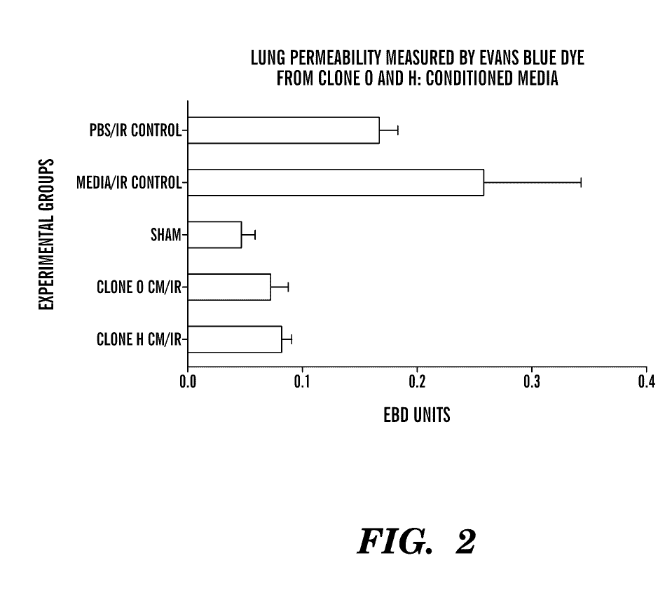

FIGS. 3A-3D show BAL cell differentials from rodents receiving MSC or conditioned media at the end of study. Cytospins from BAL were assessed for total leukocytes (FIG. 3A), neutrophils (FIG. 3B), macrophages (FIG. 3C), and lymphocytes (FIG. 3D).

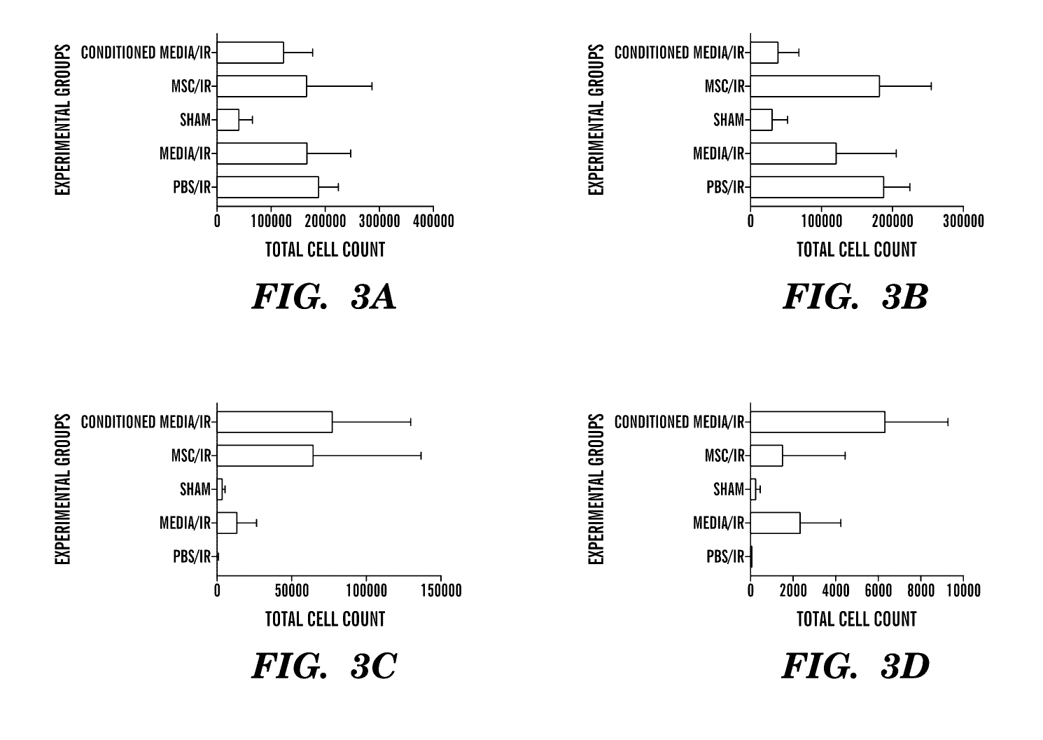

FIG. 4 shows cell differentials of peripheral blood mononuclear cells. Blood smears were assessed for neutrophils, monocytes, lymphocytes, and eosinophils. Normal range for each cell subset is marked with a bar.

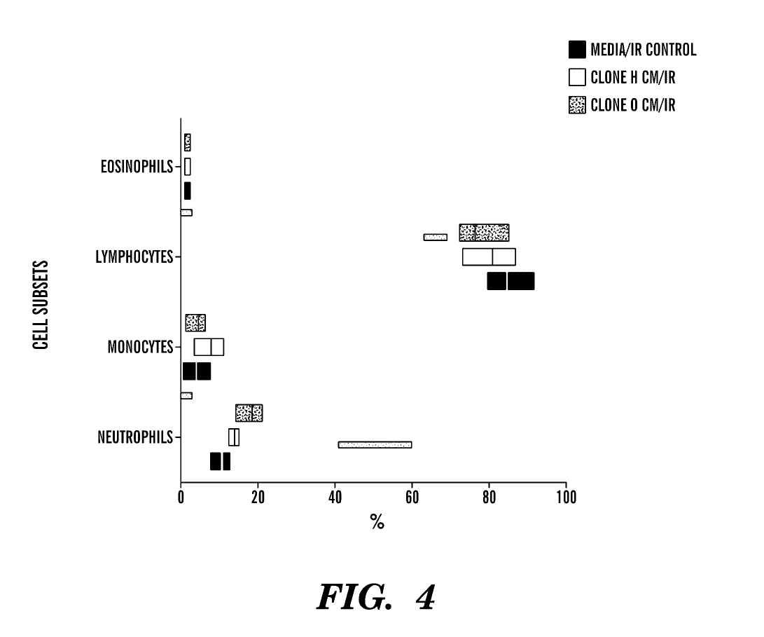

FIG. 5 shows CD3 and MAC2 immunohistochemistry staining of representative biopsies from experimental and control groups.

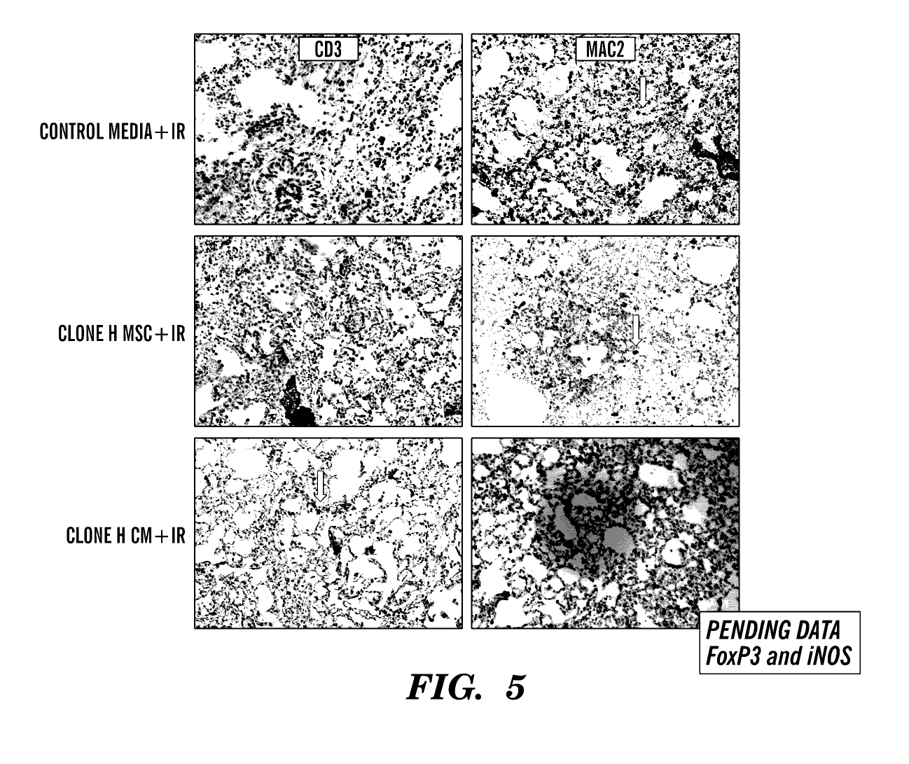

FIGS. 6A-6B. All clones were assessed for their abilities to differentiate into osteocytes and adipocytes. All clones did differentiate into osteocytes and adipocytes, under the proper set of inducing conditions. Clone O is shown as a representative sample. (FIG. 6A) Adipocytes. (FIG. 6B) Osteocytes.

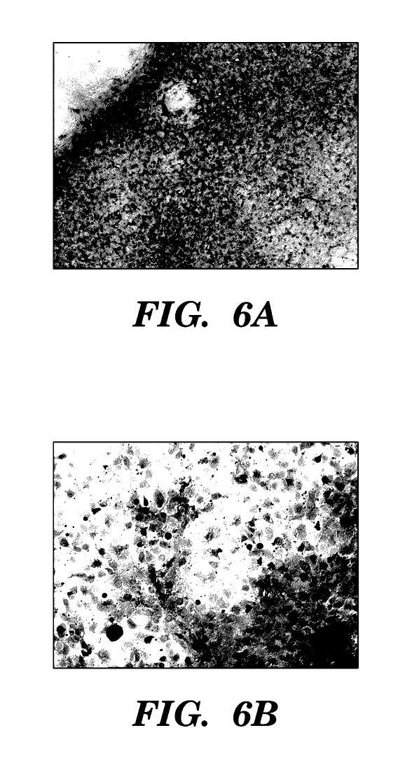

FIG. 7 shows identification of exosomes within conditioned media through electron microscopy. This is a representative sample from MSC clone O.

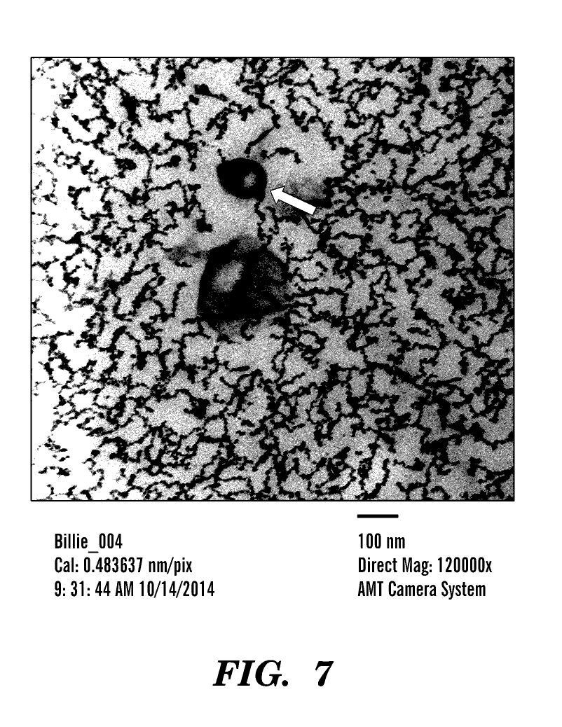

FIG. 8 shows Coomassie Blue protein detection of isolated exosomes from conditioned media. Exosomes were isolated from conditioned media from different MSC clones, lysed, and separated on a 4-12% SDS PAGE gel.

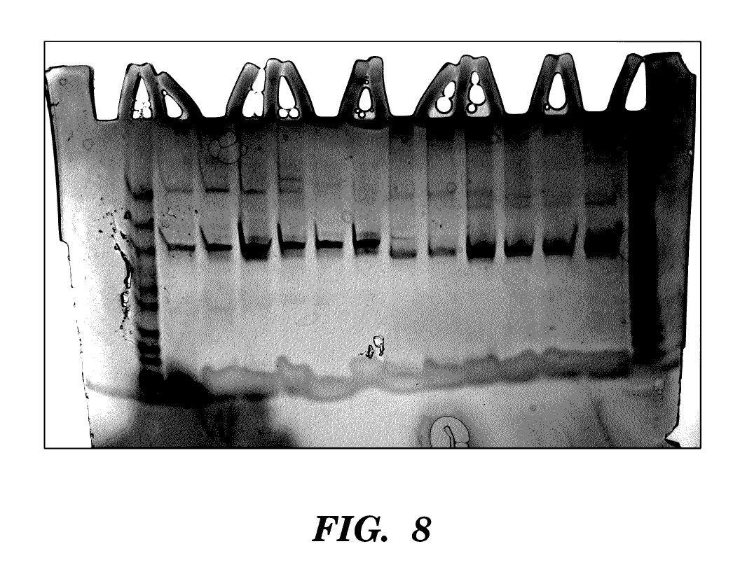

### DETAILED DESCRIPTION

As described herein, pretreatment with MSC conditioned medium prior to ischemic reperfusion injury induces ischemic tolerance. Consistent with these results, proinflammatory cytokines associated with ischemic injury were found to be significantly reduced. In addition, differentials were shown to have an increase in percentage of lymphocytes and monocytes in peripheral blood, and BAL samples showed significant increases in AV macrophages and T cells. The data indicate that protection is driven by activation and recruitment of regulatory MNC and phenotypic changes in residential cells. Data from qPCR show increases in FoxP3 expression indicating that graft infiltrating lymphocytes (GILs) are regulatory T cells. Accordingly, provided herein are “cell-free” approaches for inducing ischemic tolerance by pretreating a subject or a tissue prior to insult with conditioned medium from MSCs.

### Mesenchymal Stem Cell Conditioned Media

The MSC conditioned media for use in the methods of treating ischemic reperfusion injury and inducing ischemic tolerance described herein are obtained from cultures of mesenchymal stem cells. Mesencyhmal stem cells (MSCs) are non-hematopoietic, adherent cells that are capable of differentiating into specific types of mesenchymal or connective tissues including adipose, osseous, cartilaginous, elastic, neuronal, hepatic, pancreatic, muscular, and fibrous connective tissues. The specific differentiation pathway that these cells enter depends upon various influences including mechanical influences and/or endogenous bioactive factors, such as growth factors, cytokines, and/or local microenvironmental conditions established by host tissues.

MSCs reside in a diverse host of tissues throughout the adult organism and possess the ability to ‘regenerate’ cell types specific for these different tissues. Examples of these tissues include, but are not limited to, adipose tissue, umbilical cord blood, periosteum, synovial membrane, muscle, dermis, pericytes, blood, bone marrow and trabecular bone. Accordingly, mesenchymal stem cells for use in preparing the conditioned media described herein can be isolated or enriched from various tissues including but not limited to bone marrow, peripheral blood, blood, placenta (e.g. fetal side of the placenta), cord blood, umbilical cord, amniotic fluid, placenta, and from adipose tissue. As used herein, the term “derived from” indicates the tissue of origin of the mesenchymal stem cells.

The terms “isolate” and “methods of isolation,” as used herein, refer to any process whereby a cell or population of cells, such as a population of MSCs, is removed from a subject or sample in which it was originally found, or from a descendant of such a cell or cells. The term “isolated population,” as used herein, refers to a population of cells that has been removed and separated from a biological sample, or a mixed or heterogeneous population of cells found in such a sample. Such a mixed population includes, for example, a population of MSCs obtained from bone marrow. In some embodiments, an isolated population is a substantially pure population of cells as compared to the heterogeneous population from which the cells were isolated or enriched. In some embodiments, an isolated cell or cell population, such as a population of MSCs, is further cultured in vitro or ex vivo, e.g., in the presence of growth factors or cytokines, to further expand the number of cells in the isolated cell population or substantially pure cell population or to promote differentiation to a particular cell or cell subtype(s). Such cultures can be performed using any method known to one of skill in the art, for example, as described herein and in the Examples section.

The term “substantially pure,” with respect to a particular cell population, refers to a population of cells that is at least about 75%, at least about 80%, at least about 85%, at least about 90%, at least about 95%, at least about 98%, or at least about 99% pure, with respect to the cells making up a total cell population. In other words, the terms “substantially pure” or “essentially purified,” with regard to a population of MSCs isolated for use in the methods disclosed herein, refers to a population of MSCs that contain fewer than about 25%, fewer than about 20%, fewer than about 15%, fewer than about 10%, fewer than about 9%, fewer than about 8%, fewer than about 7%, fewer than about 6%, fewer than about 5%, fewer than about 4%, fewer than about 3%, fewer than about 2%, fewer than about 1%, or less than 1%, of cells that are not MSCs, as defined by the terms herein.

The terms “enriching” or “enriched” are used interchangeably herein and mean that the yield (fraction) of cells of one type, such as MSCs for use in the compositions and methods described herein, is increased by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 100%, by at least 150%, by at least 200%, by at least 300%, by at least 400%, by at least 500%, by at least 750%, by 1000%, or more over the fraction of cells of that type in the starting sample, culture, or preparation. Alternatively, an enriched for population of MSCs comprises at least 15%, at least 20%, at least 30%, at least 40%, at least 50%, at least 60%, at least 70%, at least 80%, at least 90%, at least 95%, at least 99%, or more of MSCs relative to other cell types.

Mesenchymal stem cells for use in generating MSC conditioned media for the methods of treating ischemic reperfusion injury and inducing ischemic tolerance described herein display specific properties or characteristics of mesenchymal stem cells. They can satisfy the morphologic, phenotypic and functional criteria commonly used to identify mesenchymal stem cells, as known in the art. The properties or characteristics can be as defined by The International Society for Cellular Therapy.

Mesenchymal stem cells for use in generating MSC conditioned media for the methods of treating ischemic reperfusion injury and inducing ischemic tolerance described herein can exhibit morphological or functional characteristics of mesenchymal stem cells including, for example, displaying a typical fingerprint whorl at confluency, forming an adherent monolayer with a fibroblastic phenotype, being capable of adhering to plastic, and/or having an average population doubling time of between 72 to 96 hours. Mesenchymal stem cells can be characterized morphologically by a small cell body with a few cell processes that are long and thin. The cell body typically contains a large, round nucleus with a prominent nucleolus, which is surrounded by finely dispersed chromatin particles, giving the nucleus a clear appearance. The remainder of the cell body contains a small amount of Golgi apparatus, rough endoplasmic reticulum, mitochondria, and polyribosomes. The cells, which are long and thin, are typically widely dispersed and the adjacent extracellular matrix is populated by a few reticular fibrils but is devoid of the other types of collagen fibrils.

Mesenchymal stem cells for use in generating MSC conditioned media for the methods described herein can be selected using combinations of one or more positive and/or negative selection markers. Methods of determining protein cell-surface expression are well known in the art. Examples include immunological methods, such as FACS analysis, as well as biochemical methods (cell-surface labeling, e.g., radioactive, fluorescence, avidin-biotin). Mesenchymal stem cells for use with the methods described herein can also be classified according to the set of standards set by the International Society for Cellular Therapy (ISCT) to define MSCs. Cultured MSCs also typically express on their surface CD73, CD90 and CD105, while lacking the expression of CD11b, CD14, CD19, CD34, CD45, CD79a and HLA-DR surface markers.

Accordingly, as used herein, a “mesencyhmal stem cell” refers to an adherent, immune privileged cell of non-hematopoietic origin that is positive for expression of at least one of CD105, CD90, CD73, and CD106, and negative for expression of at least one of CD45, CD34, CD14, CD19, CD11b, and HLA-DR, and can undergo osteogenic, adipogenic and chondrogenic differentiation ex vivo. In certain embodiments, an MSC is positive for at least one of CD105, CD90, CD73, and CD106, and negative for at least two of, at least three of, at least four of, at least five of, or even all six of CD45, CD34, CD14, CD19, CD11b, and HLA-DR. In certain embodiments, an MSC is positive for at least one, at least two of, at least three of, or even all four of CD105, CD90, CD73, and CD106, and negative for at least one of CD45, CD34, CD14, CD19, CD11b, and HLA-DR.

In some embodiments of the methods described herein, MSCs that are at a minimum positive for expression of CD105 and are negative for expression of CD34 are used to generate the MSC-conditioned medium.

In some embodiments of the methods described herein, MSCs that are at a minimum positive for expression of CD105, CD90, and CD73 and are negative for expression of CD45, CD34, and CD11b are used to generate the MSC-conditioned medium.

In some embodiments of the methods described herein, MSCs used to generate MSC conditioned medium fulfill the criteria set forth by the Mesenchymal and Tissue Stem Cell Committee of the International Society for Cellular Therapy: (i) MSCs are plastic-adherent when maintained in standard culture conditions; (ii) MSCs express CD105, CD73 and CD90, and lack expression of CD45, CD34, CD14 or CD11b, CD79alpha or CD19 and HLA-DR surface molecules; and (iii) MSCs can differentiate to osteoblasts, adipocytes and chondroblasts in vitro.

In some embodiments of the methods described herein, MSCs used to generate MSC conditioned medium suppress IR injury, as assayed using any of the methods described herein or known to the ordinary skilled artisan.

In some embodiments of the methods described herein, the mesenchymal stem cells are derived, isolated, or obtained from a tissue of a newborn human.

When transplanted, MSCs exert their effect on other cells, in part, through multiple secreted bioactive factors such as cytokines, growth factors and angiogenic factors (see Wang et al, J Hemat Oncol. 2012, 5:19). Cultured mesenchymal and mesenchymal stem cells secrete these factors into the culture medium, endowing the medium with useful properties as a supplement to cell culture, as a therapeutic composition or source thereof, as described herein. For example, U.S. Pat. No. 6,642,048 discusses a cell-free conditioned medium from MSC cell culture for feeder-layer free culture of pluripotent stem cells, but requires the transfection of hESCs. US Patent Application No. 20120251489 discusses the preparation and use of a cell-free conditioned medium from liver stem cells for inhibition of proliferation of tumor cells. US Patent Application No. 20110262392 discusses the production and use of a cell-free conditioned medium from a particular population of cultured adherent bone marrow cells for modulation of apoptosis in cancer cells. US Patent Application No. 20100323027 discusses the production of a cell-free conditioned medium from cultures of mesenchymal stem cells grown with added FGF 2 (basic FGF), for a variety of therapeutic uses. U.S. Pat. Nos. 8,815,588; 8,962,318; and 9,029,146, and U.S. Patent Application Nos. 20080219957; 20140004601; 20140031256; 20150125950; and 20150024011 also discuss the production and derivation of messenchymal stem cells, messenchymal stem cell exosomes, and conditioned media derived from MSCs. Additional studies have indicated, for example, the potential of mesenchymal stem cell conditioned medium for inhibiting lung fibrosis in pulmonary disease (Cargnoni et al, Cytotherapy 2012; 14: 153-61), enhancing kidney repair in kidney disease (van Koppen et al, PLoS one 2012; 7:1-12), stimulating angiogenesis and fracture repair in diabetic rats (Wang et al, J. Tissue Eng Regen Med 2012; 6:559-69), promotion of wound healing (Yew et al Cell Transplantation, 2011; 20:693-706) and reduction of infarct size in MI (Gnecchi et al, Meth Mol Biol 2009; 482:281-94).

Methods for increasing proliferation and survival in MSCs have been widely studied over the past few years and many factors have been proposed for increasing the expansion efficiency of these cells. Thus, one of skill in the art can prepare MSC conditioned medium as necessary for the methods described herein. The basic requirements are culture of MSCs under conditions that promote or maintain expansion while preserving the ability of the cells to differentiate along each of the adipogenic, chondrogenic, and osteogenic lineages. Different culture conditions produce mesenchymal stem cells that condition the medium in different ways. For example, many protocols relating to the expansion of MSCs include culturing in the presence of basic fibroblast growth factor (b-FGF) (Vet Res Commun. 2009 December; 33(8):811-21). It has been shown that b-FGF not only maintains MSC proliferation potential, it also retains osteogenic, adipogenic and chondrogenic differentiation potentials through the early mitogenic cycles. Vascular endothelial growth factor (VEGF) has also been shown to increase MSC proliferation (Pons et al., Biochem Biophys Res Commun 2008, 376:419-422). Hepatocyte growth factor (HGF) has been shown to affect proliferation, migration and differentiation (Furge et al., Oncogene 2000, 19:5582-5589). Platelet derived growth factor (PDGF) has been shown to be a potent mitogen of MSCs (Kang et al., J Cell Biochem 2005, 95:1135-1145). Epidermal growth factor (EGF) and heparin-binding EGF have both been shown to promote ex vivo expansion of MSCs without triggering differentiation into any specific lineage (Tamama et al., Stem Cells 2006, 24:686-695; Krampera et al., Blood 2005, 106:59-66). In addition to its mitogenic effect on MSCs, EGF also increases the number of colony-forming units by 25% (Tamama et al., J Biomed Biotechnol 2010, 795385). Addition of Wnt3a by activating the canonical Wnt pathway increased both proliferation and survival while preventing differentiation into the osteoblastic lineage in MSCs (Boland et al., J Cell Biochem 2004, 93:1210-1230).

Other growth factors that can be found in conditioned medium are known to cause mesenchymal stem cells to differentiate into specific lineages, and are contemplated for use in some embodiments of the methods described herein. Transforming growth factor beta (TGFβ), for example, is known to influence cells from the chondrogenic lineage in vivo, promoting initial stages of mesenchymal condensation, prechondrocyte proliferation, production of extracellular matrix and cartilage-specific molecule deposition, while inhibiting terminal differentiation (Bonewald et a., J Cell Biochem 1994, 55:350-357; Longobardi L, J Bone Miner Res 2006, 21:626-636). BMP-3, another member of the transforming growth factor beta family, known to enhance bone differentiation was shown to increase MSC proliferation threefold (Stewart A et al., Cell Physiol 2010, 223:658-666).

As demsontrated herein, the inventors have discovered that pretreatment of a donor subject or a donor transplant tissue with MSC conditioned medium prior to ischemic reperfusion injury induces ischemic tolerance. Accordingly, provided herein are mesencymal stem cell conditioned media for use in the methods for treating, preventing, or reducing the risk of ischemia reperfusion injury described herein.

As used herein, the terms “culture medium,” “tissue culture medium,” “growth medium” or “basal medium” refers to a solution (or component mixtures in powder form) of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cells' survival. These are compounds that the cells themselves cannot synthesize, due to the absence of one or more of the gene(s) that encode the protein(s) necessary to synthesize the compound (e.g., essential amino acids) or, with respect to compounds which the cells can synthesize, but because of their particular developmental state the gene(s) encoding the necessary biosynthetic proteins are not being expressed at sufficient levels. A number of growth media are known in the art of mammalian cell culture, such as, for example, Dulbecco's Modified Eagle Media (DMEM), Knockout-DMEM (KO-DMEM), and DMEM/F12. Culture media can also comprise reagents that are usually found used in cell culture, such as antibiotics (e.g., gentamycin and kanamycin), albumin, and serum, although serum-free medium is specifically contemplated. In some embodiments of the aspects described herein, a tissue culture medium can comprise autologous plasma (i.e., the MSCs being cultured and the autologous plasma are collected from the same body). In some embodiments, the growth medium can comprise serum from the transplant recipient. In some embodiments, the growth medium can comprise serum from the transplant donor.

In some embodiments, MSCs can be grown in minimal media, such as RPMI, comprising 1-5% serum, such as transplant recipient serum, transplant donor serum, or fetal bovine serum and further comprises antibiotics, such as penicillin and streptomycin.

As used herein, the term “MSC conditioned medium” or “mesenchymal stem cell conditioned medium” refers to a growth medium that comprises soluble factors (“culture-derived growth factors”) derived from mesenchymal stem cells, preferably human mesenchymal stem cells, cultured in the medium. In some embodiments of the methods described herein, the MSC conditioned medium is growth media conditioned by the growth of bone marrow or adipose-derived mesenchymal stem cells, such as, human mesenchymal stem cells.

Techniques for culturing and isolating conditioned medium from a cell culture are known in the art. For example, in some embodiments, the population of MSC cells are cultured (in vitro or ex vivo) on polystyrene plastic surfaces (e.g., in a flask) so as to enrich for mesenchymal stem cells by removing non-adherent cells (i.e., non-mesenchymal stem cells). Thus, according to some embodiments of the aspects described herein, the mesenchymal stem cells for use with the methods described here are “adherent” or “plastic-adherent” cells (e.g., cells remaining adhered to the plastic surface after removal of non-adherent, non-mesenchymal cells).

In some embodiments, MSCs for use in generating MSC conditioned medium are cultured in the absence of co-culture. As used herein, the term “co-culture” refers to a mixture of two or more different kinds of cells that can be grown together, for example, stromal feeder cells and MSCs. Thus, in some embodiments, the MSCs can be cultured as a monolayer, i.e., in the absence of any feeder cells.

In some embodiments, MSCs for use in generating MSC conditioned medium are cultured in a serum-free medium. The term “serum-free medium,” as used herein, refers to cell culture media which is free of serum. Serum-free media are known in the art, and are described for example in U.S. Pat. Nos. 5,631,159 and 5,661,034. Serum-free media are commercially available from, for example, Gibco-BRL (Invitrogen). In some embodiments, the serum-free medium can be protein free, in that it lacks proteins, hydrolysates, and components of unknown composition. The serum-free medium can comprise chemically-defined medium in which all components have a known chemical structure. Chemically-defined serum-free medium can be used as it provides a completely defined system which eliminates variability allows for improved reproducibility and more consistent performance, and decreases possibility of contamination by adventitious agents. An exemplary serum-free medium is Knockout DMEM medium (Invitrogen-Gibco, Grand Island, N.Y.). Serum-free medium can be supplemented with a serum-replacement composition of known or defined composition, thereby avoiding use of non-autologous serum. In some embodiments, serum-free medium can be supplemented with one or more components, such as serum replacement medium, at a concentration of for example, 5%, 10%, 15%, etc. The serum-free medium can, for example, be supplemented with 10% serum replacement medium from Invitrogen-Gibco (Grand Island, N.Y.). In some embodiments of the aspects described herein, a culture medium is supplemented with autologous serum, i.e., from the transplant recipient. In some embodiments of the aspects described herein, a culture medium is supplemented with serum from the transplant donor, provided the transplant recipient and donor are tissue-matched.

In some embodiments of the methods described herein, the MSC conditioned medium is essentially cell-free. As used herein, “essentially cell-free” refers to a MSC conditioned medium that contains fewer than about 10%, fewer than about 5%, fewer than about 1%, fewer than about 0.1%, fewer than about 0.01%, fewer than about 0.001%, fewer than about 0.0001%, or less, than the number of cells per unit volume, as compared to the MSC culture from which it was separated. As used herein, the term “MSC conditioned medium” also encompasses such a medium that has been treated by concentration, filtration, extraction, fractionation or other means for preserving, increasing the potency, improving the stability, removing impurities, etc. Thus, MSC conditioned medium includes extracts and fractions of a conditioned medium that retains the ability to protect against ischemic reperfusion injury in the models described herein.

As understood by one of ordinary skill in the art, in some embodiments, the MSC conditioned medium for use in the methods described herein can also include additional components added after isolation and collection of the MSC conditioned medium, such as preservatives, anti-bacterial and antifungal agents, nutrients, biologically active agents such as cytokines and chemokines, drugs, etc. In some embodiments, the MSC conditioned medium comprises one or more growth factors. A number of growth factors are known in the art, including, for example, PDGF, EGF, TGF-a, FGF, FGF2, NGF, Erythropoietin, TGF-b, IGF-I and IGF-II. Accordingly, in some embodiments, the MSC conditioned medium comprises one or more growth factors selected from PDGF, EGF, heparin binding EGF, TGF-a, FGF, FGF2/b-FGF, VEGF, NGF, Erythropoietin, Wnt3a, TGF-b, IGF-I and IGF-II.

Still further, in some embodiments, the MSC conditioned medium can be processed by heating, for example, pasteurization. In some embodiments, the MSC conditioned medium can be stored as is, refrigerated or frozen. In some embodiments, the MSC conditioned medium is stored frozen, at about −5° to about −80° C. In some embodiments, the MSC conditioned medium is dehydrated (e.g., desiccated, lyophilized, etc) and stored dry, and reconstituted at desired concentration (for example, with water) before use. Further, MSC conditioned media can be tested for the presence of contaminants, such as endotoxin.

Cells in culture will generally continue growing until confluence, when contact inhibition causes cessation of cell division and growth. Such cells can then be dissociated from the substrate or flask, and “split” or passaged, by dilution into tissue culture medium and replating. The cultured cells can therefore be passaged, or split during culture. They may be split at a ratio of 1:2 or more, such as 1:3 or 1:4, 1:5 or more. The term “passage” designates the process involving taking an aliquot of a confluent or near confluent culture, inoculating into fresh medium, and in culturing the cells or cell line until confluence or saturation is obtained.

The mesenchymal stem cells derived according to the methods described here can be maintained for a large number of generations and maintain self-renewal without the requirement for transformation. Thus, for example, known transformation treatments such as fusion with immortalised cells such as tumour cells or tumour cell lines, viral infection of a cell line with transforming viruses such as SV40, EBV, HBV or HTLV-1, transfection with specially adapted vectors, such as the SV40 vector comprising a sequence of the large T antigen (R. D. Berry et al., Br. J. Cancer, 57, 287-289, 1988), telomerase (Bodnar-A-G. et. al., Science (1998) 279: p. 349-52) or a vector comprising DNA sequences of the human papillomavirus (U.S. Pat. No. 5,376,542), introduction of a dominant oncogene, or by mutation are therefore not required in the methods described here for making mesenchymal stem cell conditioned medium.

Accordingly, in some embodiments, mesenchymal stem cells for generating the MSC conditioned mediua described herein can be propagated without transformation for more than 50 generations. In some embodiments, the mesenchymal stem cells can be propagated indefinitely and without transformation as mesenchymal stem cell lines.

In some embodiments of the methods described herein, the MSC conditioned medium is collected following removal of growth medium from the mesenchymal stem cell culture and replacement with fresh medium. In some embodiments of the methods described herein, the MSC conditioned medium is collected at least about 12 hours following replacement of the medium, about 24 hours, about 36 hours, about 48 hours, about 60 hours or more following replacement of the medium. In some embodiments, the MSC conditioned medium is collected 24 or 48 hours following replacement. In some embodiments, the medium is replaced, and MSC conditioned medium collected just before a passage of the cells, for example, before the 1^{st} passage, before the 2^{nd} passage, before the 3^{rd} passage, before the 4^{th} passage, or more. In some embodiments, the MSC conditioned medium is replaced and MSC conditioned medium for use in the methods described herein is collected before the 3^{rd} or before the 4^{th} passage.

In some embodiments of the methods described herein, MSCs are allowed to become no more than about 75% confluent, no more than about 80% confluent, no more than about 85% confluent, or no more than about 90% confluent before harvesting the MSC conditioned media.

In some embodiments, the medium used for replacement prior to collection of MSC conditioned medium is not identical to the growth medium used during the previous culture period. In some embodiments, the replacement medium provided prior to collection of MSC conditioned medium (and thus the MSC conditioned medium) is serum-free medium. Thus, MSCs can be expanded or maintained in serum-containing medium and then switched to serum-free medium, or medium supplemented with transplant recipient or transplant donor serum prior to collection of conditioned medium for use in the methods described herein.

In some embodiments, MSC conditioned media obtained from MSC cultures are centrifuged, for example at 1000 rpm, to remove any residual cellular debris, and then stored frozen until used.

In some embodiments, MSC conditioned media for use in the methods described herein are tested for the presence of one or more cytokines, such as TNFα and IL1β. Preferably, the MSC conditioned media used in the methods described herein are selected to have low levels of TNFα and IL1β.

In some embodiments, MSC conditioned media for use in the methods described herein are analyzed for the presence and/or protein content of their exosomes. As used herein, “exosomes” refer to 50-150 nm microvesicles that contain various cytokines, miRNA, and other proteins that can elicit an immune response. Exosomes are found in various bodily fluids and have also been identified as being secreted into culture media, and are secreted by different cell subsets, including MSCs. Exosomes found in MSC conditioned media can have a density of about 1.13-1.19 g/ml; can float on sucrose gradients; can be enriched in cholesterol and sphingomyelin, and lipid raft markers, such as GM1, GM3, flotillin and the src protein kinase Lyn; can comprise one or more proteins present in MSCs or MSC conditioned medium; and/or can comprise RNA, for example mRNA or miRNA.

MSC exosomes can be responsible for at least one activity of the MSC conditioned medium useful in the methods described herein. MSC exosomes can be responsible for, and carry out, substantially most or all of the functions of the MSC conditioned medium. For example, the MSC exosomes can be a substitute, i.e., a biological substitute, for the MSC conditioned medium, in some embodiments. Such MSC exosomes, and combinations of any of the molecules comprised therein, including, in particular, proteins or polypeptides, can be used, in some embodiments, to supplement the activity of, or in place of, the MSC conditioned medium for the methods described herein of treating, preventing, or moderating ischemia reperfusion injuries. In such embodiments, the MSC exosomes preferably have at least one property of the MSC conditioned medium, for example, a therapeutic or restorative activity of the MSC conditioned medium.

For those embodiments where MSC exosomes are used in place of, or to supplement MSC conditioned medium, the MSC exosomes can be produced or isolated in a number of ways known to those of ordinary skill in the art. For example, MSC exosomes can be isolated by being separated from non-associated components based on any property of the MSC exosome including, but not limited to, molecular weight, size, shape, composition and/or biological activity. For example, MSC conditioned medium can be filtered or concentrated or both during, prior to or subsequent to separation. The MSC conditioned medium, optionally filtered or concentrated or both, can be subject to further separation means, such as column chromatography. One or more properties or biological activities of the MSC exosomes can be used to track their activity during fractionation of the MSC conditioned medium.

### Methods of Treating Ischemia Reperfusion Injuries

As demonstrated herein, administration of mesenchymal stell cell conditioned medium to a donor subject or transplant tissue prior to transplant induces ischemic tolerance. Consistent with these data, proinflammatory cytokines associated with ischemic injury were significantly reduced. Blood differentials showed an increase in percentage of lymphocytes and monocytes in peripheral blood. BAL samples showed significant increases in AV macrophages and T cells. The data indicate that protection from ischemia reperfusion injury is driven by activation and recruitment of regulatory MNC and phenotypic changes in residential cells. Data from qPCR show increased FoxP3 expression and indicate that graft infiltrating lymphocytes (GILs) include regulatory T cells.

Current treatment strategies for ischemia reperfusion of the lung include, for example, lung protective ventilation, fluid management, optimization of organ preservation, reduction in ventilation and/or anoxic ischemic time, and/or inhaled nitric oxide. In addition, current investigational studies have focused on complement inhibitors, such as inhibition of C3 and C5 pathways; inhibition of sterile immunity, such as AV macrophage depletion and antibody target inhibition, and gene therapy.

In contrast, the methods described herein use MSC conditioned media administered to a transplant tissue, either via administration to a transplant donor or via ex vivo administration, to protect the transplant tissue from the effects of ischemic injury, thereby reducing the effects and consequences of ischemia reperfusion injury when the transplant tissue is transplanted into a transplant recipient.

Accordingly, provided herein, in some aspects are methods of preventing, reducing the severity of, or reducing the risk of ischemia reperfusion injury to a transplant tissue comprising treating a transplant tissue with a therapeutically effective amount of an MSC (mesenchymal stem cell)-conditioned medium prior to transplanting the tissue to a transplant recipient.

As used herein, the term “ischemia-reperfusion injury” refers to an injury resulting from the restoration of blood flow to an area of a tissue or organ that had previously experienced deficient blood flow due to an ischemic event. As used herein, the terms “ischemic event,” “injury resulting from ischemia,” “injury caused by ischemia,” and “ischemic injury” refer to an injury to a cell, tissue, or organ caused by ischemia, or an insufficient supply of blood, and thus oxygen, thereby resulting in damage or dysfunction of the tissue or organ (Piper, H. M., Abdallah, C., Schafer, C., Annals of Thoracic Surgery 2003, 75:644; Yellon, D. M., Hausenloy, D. J., New England Journal of Medicine 2007, 357:1121). Injuries that result from ischemia can affect various tissues and organs and include pulmonary ischemia, cardiovascular ischemia, cerebrovascular ischemia, renal ischemia, hepatic ischemia, ischemic cardiomyopathy, cutaneous ischemia, bowel ischemia, intestinal ischemia, gastric ischemia, pancreatic ischemia, skeletal muscle ischemia, abdominal muscle ischemia, limb ischemia, ischemic colitis, mesenteric ischemia and silent ischemia. Thus, an injury resulting from ischemia can affect, for example, a lung, heart, kidney, liver, brain, muscle, intestine, stomach, or skin.

Oxidative stresses associated with reperfusion can cause damage to the affected tissues or organs. “Ischemia-reperfusion injury,” is a type of ischemic event that is characterized biochemically by a depletion of oxygen during an ischemic event involving interrupted blood flow, followed by reoxygenation and the concomitant generation of reactive oxygen species during reperfusion (Piper, H. M., Abdallah, C., Schafer, C., Annals of Thoracic Surgery 2003, 75:644; Yellon, D. M., Hausenloy, D. J., New England Journal of Medicine 2007, 357:1121).

An ischemia reperfusion injury can be caused, for example, by a natural event (e.g., restoration of blood flow following a myocardial infarction), a trauma, or by one or more surgical procedures or other therapeutic interventions that restore blood flow to a tissue or organ that has been subjected to a diminished supply of blood. Such surgical procedures include, for example, tissue or organ transplants, including, but not limited to, lung transplantation, coronary artery bypass graft surgery, coronary angioplasty, and the like.

Accordingly, in some embodiments, the methods described herein are useful for the prevention, treatment, and/or reduction of risk of ischemia reperfusion injury in a transplant tissue. As used herein, “transplant tissue” refers to any tissue or organ or portion thereof to be transplanted to a transplant recipient, including, but not limited to, a lung or a portion or lobe thereof, a kidney, a heart, a liver or a portion thereof, a pancreas or a portion thereof, a bone, bone marrow, or a segment of bowel or other portion of the alimentary canal. In some embodiments, the transplant tissue is a lung or a portion or lobe thereof.

As used herein, a “transplant donor” is a subject from whom a transplant tissue or organ is obtained prior to transplantation of the tissue to a transplant recipient. The methods described herein can be applied to a transplant tissue prior to removal from the donor (i.e., in situ), or following removal or excision from the donor, but prior to transplantation into the recipient. As used herein, “excision” or “removal,” as applied to a transplant tissue or organ, refer to the physical removal of the tissue or organ or portion thereof from the transplant donor. Such removal includes any surgical or non-surgical procedure by which the transplant tissue is removed and includes conventional surgical procedures, as well as laparoscopic interventions.

As used herein, a “transplant recipient” is a subject into whom a transplant tissue or organ is placed after excision or removal of the tissue from the transplant donor.

In some embodiments of the methods described herein for treatment of ischemia-reperfusion injuries, the therapeutically effective amount of the MSC conditioned medium is administered to a transplant donor prior to excision of the transplant tissue or organ.

The MSC conditioned medium can be administered to the transplant donor prior to the excision or surgical removal of the transplant tissue. For example, the MSC conditioned medium can be administered to the transplant donor about 5 minutes before the excision of the transplant tissue, about 10 minutes, about 15 minutes, about 30 minutes, about 45 minutes, about 1 hour, about 2 hours, about 3 hours, about 4 hours, about 5 hours, about 12 hours, about 24 hours, or about 48 hours prior to excision of the transplant tissue. In some embodiments of the methods described herein, the transplant donor is administered the therapeutically effective amount of the MSC-conditioned medium for between 15 minutes to 1 hour prior to excision of the transplant tissue from the transplant donor. In some embodiments of the methods described herein, the transplant donor is administered the therapeutically effective amount of the MSC-conditioned medium for at least 30 minutes prior to excision of the transplant tissue from the transplant donor.

In some embodiments of the methods described herein for treatment of ischemia-reperfusion injuries, treating comprises perfusing the transplant tissue with the therapeutically effective amount of the MSC-conditioned medium ex vivo prior to transplanting the transplant tissue to the transplant recipient. For example, prior to transplant of a transplant tissue or organ into transplant recipient (e.g., during storage or transport of the tissue or organ in a sterile environment, or immediately following excision of the tissue from the transplant donor), the transplant tissue or organ can be contacted or perfused with the MSC conditioned medium (e.g., bathed in a solution comprising the MSC conditioned medium) to inhibit ischemia-reperfusion injury following transplant.

In some embodiments of the methods described herein, the transplant donor is administered the therapeutically effective amount of the MSC-conditioned medium prior to removal of the transplant tissue and the transplant tissue is then maintained in or contacted with a composition comprising MSC conditioned medium until the transplant tissue or organ is transplanted into a transplant recipient. Once transplanted into the recipient, the transplant tissue can, in some embodiments, be further contacted with a therapeutically effective amount of MSC conditioned medium directly or indirectly (e.g., by i.v. administration to the transplant recipient).

The phrase “preventing, reducing the severity of, or reducing the risk of ischemia reperfusion injury to a transplant tissue” (or variations thereof) generally refers to decreasing or reducing the degree of injury to a transplant tissue or organ caused by a depletion of oxygen during an ischemic event followed by reoxygenation and the concomitant generation of reactive oxygen species during reperfusion. Such reduction of injury to a transplant tissue or organ can be measured, for example, by a decrease in proinflammatory cytokines associated with ischemic injury, or an increase in numbers or functions of regulatory T cells at the site of the transplant tissue or organ. The Evans Blue Dye assay described herein can be used to quantify lung injury based on lung permeability, for those embodiments related to lungs or portions thereof as transplant tissue. For the avoidance of doubt, the terms “decrease,” “inhibit” “reduce” and variations thereof mean a decrease, as compared to a reference level, of at least 10% or greater, 15% or greater, 20% or greater, 30% or greater, 40% or greater, 45% or greater, more preferably at least 50% or greater, 55% or greater, 60% or greater, 65% or greater, 70% or greater, and most preferably at least 75% or greater, 80% or greater, 85% or greater, 90% or greater, or 95% or greater, for a given parameter, in a transplant donor or transplant tissue treated with a therapeutically effective amount of a MSC conditioned medium relative to a transplant donor or transplant tissue in the absence of the MSC conditioned medium.

As used herein, in regard to any of the compositions, methods, and uses described herein, the terms “treat” “treatment” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein, the phrase “therapeutically effective amount” refers to an amount of the MSC-conditioned medium effective to treat a disease or disorder in a mammal. The term “effective amount” as used herein refers to the amount of MSC-conditioned medium needed to alleviate at least one or more symptom of ischemic reperfusion injury, and relates to a sufficient amount of the MSC-conditioned medium required to provide the desired effect. An effective amount as used herein would also include an amount sufficient to delay the development of a symptom of ischemic reperfusion injury, alter the course of a symptom of ischemic reperfusion injury (for example but not limited to, slow the progression of a symptom of ischemic reperfusion injury), or reverse a symptom of ischemic reperfusion injury. Thus, it is not possible to specify the exact “effective amount”. However, for any given case, an appropriate “effective amount” can be determined by one of ordinary skill in the art using only routine experimentation. Effective amounts, toxicity, and therapeutic efficacy can be determined by standard pharmaceutical procedures in experimental animals. A therapeutically effective dose can be estimated initially using, for example, animal models, as described herein.

Compositions comprising MSC conditioned medium described herein can be administered to a subject in need thereof by any appropriate route which results in an effective treatment in the subject. As used herein, the terms “administering,” “delivering,” and “introducing” are used interchangeably and refer to the placement of MSC conditioned medium described herein into a transplant donor or transplant tissue by a method or route which eventually results in partial localization of such MSC conditioned medium at a desired site, such that a desired effect(s) is produced. Suitable routes of administration can, for example, include oral, rectal, transmucosal, especially transnasal, intestinal, or parenteral delivery, including intramuscular, subcutaneous, and intramedullary injections, as well as intrathecal, direct intraventricular, intravenous, intraperitoneal, intranasal, or intraocular injections. Alternatively, one can administer MSC conditioned medium in a local rather than systemic manner, for example, via injection of the MSC conditioned medium directly into a tissue region of a transplant donor, such as via intratracheal administration. The therapeutically effective amount of the MSC conditioned medium described herein can be administered to a transplant donor or to the transplant tissue by any suitable means known in the art for delivering fluid into the body, and includes injection, surgical drips, catheters (including perfusion catheters), such as those described in U.S. Pat. No. 6,139,524, for example, drug delivery catheters, such as those described in U.S. Pat. No. 7,122,019. Delivery to the lungs or nasal passages, including intratracheal or intranasal delivery, can be achieved using for example a nasal spray, puffer, inhaler, etc as known in the art (for example as shown in U.S. Design Pat. No. D544,957. Delivery to the kidneys can be achieved using an intra-aortic renal delivery catheter, such as that described in U.S. Pat. No. 7,241,273.

As understood by one of ordinary skill in the art, it will be evident that the particular delivery system for administration should be configurable to deliver the required therapeutically effective amount of the MSC conditioned medium, at the appropriate interval(s), in order to achieve optimal treatment. Also, as understood by one of ordinary skill in the art, the method of administration used, whether systemic or local or both, depends on the particular target transplant tissue or organ to which the MSC conditioned medium is to be administered, and the skilled person will be able to determine which means to employ accordingly.

For those embodiments where MSC exosomes are used in place of, or to supplement MSC conditioned medium, the MSC exosomes can be delivered to the human or animal body by any suitable means. The delivery system used can comprise a source of MSC exosomes, such as a container containing the MSC exosomes. The delivery system can comprise a dispenser for dispensing the MSC exosomes to a target. It will be evident that the delivery method used depends on the particular organ to which the MSC exosomes are to be delivered, and the skilled person will be able to determine which means to employ accordingly. For example, any variety of catheter, or a perfusion catheter, can be used to administer the MSC exosomes. Alternatively the MSC exosomes can be coated or impregnated on a stent that is placed in a blood vessel specific for the tissue or organ to be transplanted.

Pharmaceutically acceptable carriers and diluents include saline, aqueous buffer solutions, solvents and/or dispersion media. The use of such carriers and diluents is known in the art. The solution is preferably sterile and fluid.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Materials and Methods

### Lung Ischemia Reperfusion Model

### Animals

Pathogen-free Long Evans rats (weight 250-275 g; Harlan Sprague-Dawley, Indianapolis, Ind.) were used for all experiments in a well-established warm in situ ischemia reperfusion model.

### Procedure

Rodents were anesthetized with 2.5% isoflurane and a 14-gauge angiocatheter inserted into the trachea through a midline neck incision. Rats were connected to a CWE ventilator (CWE Inc, Ardmore, Pa.) and settings were maintained at an inspired oxygen content of 60% with a positive end expiratory pressure of 2 cm H₂O and respiratory rate of 80 breaths/min. A left thoracotomy was performed and the left lung mobilized atraumatically. Heparin (50U) was administered through the penile vein. After 5 minutes of circulation, a non-crushing clamp was placed across the left lung hilum. The clamp was removed after 90 minutes and the left lung reventilated and reperfused for 4 hours. A midline laparotomy and sternotomy was performed and animals were euthanized by aortic transection. The heart-lung block was excised and the pulmonary circulation flushed with 20 mL of phosphate buffered saline (PBS).

### Mesenchymal Stromal Cells (MSC)

Clonal rat MSC lines were generated by Beverly Torok-Storb at Fred Hutchison Cancer Research Center. Preliminary studies interrogated 4 of the 8 clones that were provided. All MSC lines differ in morphology, function, surface phenotype, and gene expression. Media used to grow these cells included RPMI, 5% penstrep, and 10% FBS. MSC preparation for in vivo experiments included 2 minutes of 0.25% trypsin, followed by a series of washes and centrifugation steps at 1,200 rpm for 10 minutes at room temperature. Cells were counted and resuspended in a volume of 200 uL with a final concentration of 1.0×10⁶ cells.

### Conditioned Media

Different clonal rat MSC lines were expanded between passages 6-12 and conditioned media collected for in vivo and in vitro experiments. Conditioned media were centrifuged at 1,200 rpm for 10 minutes at 4° C. to remove any cells and debris. A final volume of 200 uL was infused intracheal (IT) in experimental rodents.

### Study Cohorts

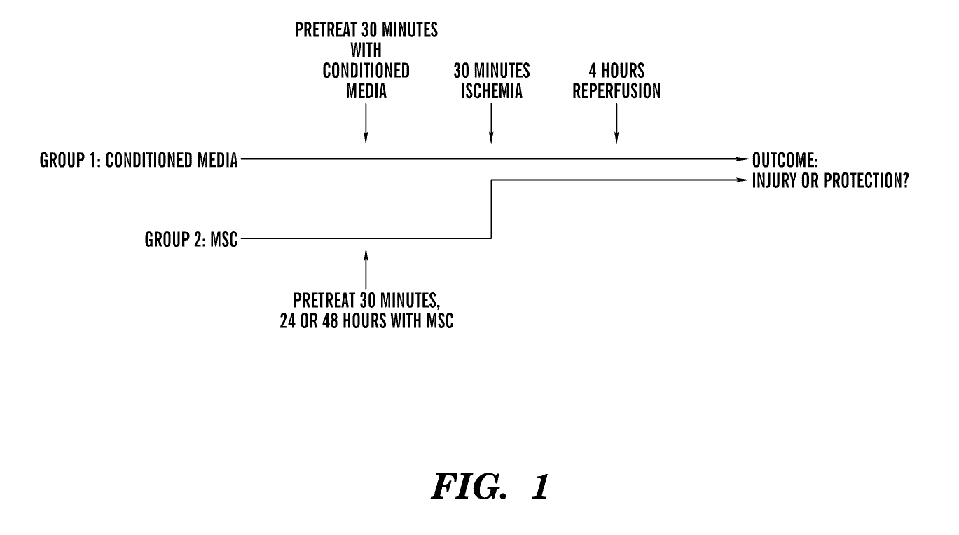

Eleven cohorts were studied (Table 1). Negative control animals received SHAM but did not undergo ischemia reperfusion (IR). Two positive control groups were used for these studies, either receiving PBS or media prior to undergoing IR. The remaining eight cohorts received either MSC or conditioned media from 4 different clones. Conditioned media were infused intracheally, 30 minutes prior to undergoing IR. MSC studies were done for three different timepoints: 30 minutes, 24 hours, and 48 hours prior to IR (FIG. 1).

TABLE 2
Study characteristics

| | Pretreatment characteristics | | |
|---|---|---|---|
| Experimental | | Time prior | Total |
| group | Treatment¹ | to IR | number |
| Negative control | SHAM | X | 4 for |
| | | | each clone |
| Positive control | PBS/IR | X | 10 |
| Positive control | Media/IR | X | 10 |
| Clone O | MSC/IR | 30 min, 24 hrs, | 4, 4, 4 |
| | | and 48 hrs | |
| | Conditioned media/IR | 30 min | 8 |
| Clone H | MSC/IR | 30 min, 24 hrs, | 4, 4, 4 |
| | | and 48 hrs | |
| | Conditioned media/IR | 30 min | 8 |
| Clone N | MSC/IR | 30 min | 4 |
| | Conditioned media/IR | 30 min | 4 |
| Clone E | MSC/IR | 30 min | 4 |
| | Conditioned media/IR | 30 min | 4 |

¹All treatments were adminstered intratracheal (IT).

### Lung Permeability Assessment

Animals received Evans Blue Dye at a dose of 20 mg/kg of body weight intravenous 30 minutes before the end of the reperfusion. After the reperfusion, a midline abdominal incision was performed and abdominal aorta and vein were severed. The left ventricle was vented with a small incision at the apex of the heart. The mitral apparatus was dilated with the left atria using a 14-gauge cannula passing through the mitral valve and into the left atrium to allow free flow of effluent blood from the lung. The pulmonary vasculature was flushed by injecting 10 mL of PBS with a 20-gauge cannula from the pulmonary artery. The left lung was excised and snapfrozen in liquid nitrogen. The frozen lung was homogenized in 2 ml PBS, diluted with 4 mL of formamide, and then incubated at 60° C. for 24 hours. The homogenate was centrifuged at 8,000 rpm for 5 minutes at room temperature. The supernatants were collected and measured by spectrophotometry at 620 nm.

### BAL Procurement

After 4 hours of reperfusion, a clamp was placed across the right hilum and the left lung lavaged with sterile PBS. The recovered fluid was centrifuged at 1,800 rpm for 10 minutes at 4° C. The supernatant was collected from each sample and stored to assess for cytokines. The cell pellet was re-suspended and lysed for red blood cells. Cells were washed, centrifuged at 1,200 rpm for 10 minutes, and re-suspended in 1 mL of PBS. Total cell counts were assessed using a hemacytometer.

### Protein Analysis for Cytokines by ELISA

BAL supernatants were processed and collected as previously described above. Supernatants were analyzed using sandwich ELISA kits (Thermoscientific, Carlsbad, Calif.) for IL10, TNFα, and IL1β cytokines. Standards and samples were run in duplicate, and well to well variation did not exceed 5%.

### Differentials

Blood smears were prepared manually on glass slides and stained with Wright Giemasa dye. Slides were manually differentiated, 100 cells per blood smear by the same reviewer and the average taken for analysis. BAL samples were centrifuged at 1200 rpm for 10 minutes at room temperature and cells resuspended in 500 uL of PBS. Cytospins were prepared from cells isolated from BAL and stained in Diff-Quik to assess for neutrophils, macrophages, and lymphocytes.

### Results

### Evans Blue Dye

As described above, 4 out of 8 clones were studied in the ischemia reperfusion injury model described herein. Conditioned media from clones O and H showed significant reduction in lung injury when compared to concurrent controls (p=0.0021 and p=0.028, respectively). Pretreatment with MSC from clone H reduced injury, while clone O showed no improvements after insult (p=0.0018 and p=0.8786, respectively). There were no differences with administration of MSC or conditioned media from clones N and E when compared to controls.

Exosomes are 50-150 nm microvesicles that contain various cytokines, miRNA, and other proteins that can elicit an immune response. Exosomes are found in various bodily fluids and most recently, identified in culture media, secreted by different cell subsets. By electron microscopy, exosomes within MSC-derived conditioned media were identified from the clones that were used herein in in vivo experiments. These data shows differences in exosomal protein content within the conditioned media from different MSC clones.

### Cytokines: IL-1β, TNF-α, and IL-10

IL-1β, TNF-α, and IL-10 cytokines were assessed in BAL supernatant from control and experimental groups was quantified by ELISA. Study cohorts receiving conditioned media from clone O and H prior to IR showed reduction in proinflammatory cytokines compared to controls. IL-10 was slightly increased. Rodents receiving clone O or H MSC show no differences in IL-1β, TNF-α, and IL-10 concentrations compared to controls. Data show differences between protective and non-protective conditioned media samples. Clones O and H (protective) had less TNFα and IL1β when compared to clones N and E (non-protective). IL-10 expression appears to be similar between the four clones.

### Cell Counts

Total leukocyte and neutrophil counts from BAL were not significantly different between controls and treated animals (FIG. 1). The total number of macrophages for both MSC and conditioned media treated groups were substantially increased. Total lymphocytes remained unchanged for MSC treated animals compared to controls but substantially increased in animals treated with conditioned medium.

Pretreatment with MSC conditioned media prior to ischemic reperfusion injury appears to induce ischemic tolerance. Proinflammatory cytokines associated with ischemic injury are significantly reduced. Interestingly, IL10 expression is not elevated in treatment groups. Differentials show an increase in percentage of lymphocytes and monocytes in peripheral blood. BAL samples showed significant increases in AV macrophages and T cells. Protection appears to be driven by activation and recruitment of regulatory MNC and phenotypic changes in residential cells. Data from qPCR show increases in FoxP3 expression and is indicative that graft infiltrating lymphocytes (GILs) are regulatory T cells. These cells could play a major role in the development of ischemic tolerance in this model.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of skill in the art, such as the Oxford Dictionary of Biochemistry and Molecular Biology (Ed. Anthony Smith, Oxford University Press, Oxford, 2004).

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

1. A method of preventing, reducing the severity of, or reducing the risk of ischemia reperfusion injury to a transplant tissue comprising treating a transplant tissue with a therapeutically effective amount of an MSC (mesenchymal stem cell)-conditioned medium prior to transplanting the tissue to a transplant recipient.

2. The method of claim 1, wherein the treating comprises administering the MSC-conditioned medium to a transplant donor prior to excision of the transplant tissue.

3. The method of claim 2, wherein the MSC-conditioned medium is administered for between 15 minutes to 1 hour prior to excision of the tissue from the transplant donor.

4. The method of claim 3, wherein the MSC-conditioned medium is administered for at least 30 minutes prior to excision of the tissue from the transplant donor

5. The method of claim 2, wherein the MSC-conditioned medium is administered to the transplant donor by a method selected from the group consisting of local administration to the tissue and systemic infusion.

6. The method of claim 5, wherein the MSC-conditioned medium is administered by intratracheal local administration.

7. The method of claim 1, wherein the treating comprises perfusing the transplant tissue with the MSC-conditioned medium ex vivo prior to transplanting the transplant tissue to the transplant recipient.

8. The method of claim 1, further comprising the step of transplanting the tissue into a transplant recipient.

9. The method of claim 1, wherein the transplant tissue is an organ comprising a lung or a lobe thereof, a kidney, a heart, a liver or a lobe thereof, a pancreas, a bone, bone marrow, or a segment of bowel or alimentary canal.

10. The method of claim 9, wherein the transplant tissue is a lung or a lobe thereof.

11. The method of claim 1, wherein the MSC-conditioned medium is free of cells.

12. The method of claim 1, wherein the MSC-conditioned medium is serum-free.

13. The method of claim 1, wherein the MSC-conditioned medium is obtained from a cell culture comprising MSCs that are positive for expression of at least one of CD105, CD90, CD73, and CD106, and are negative for expression of at least one of CD45, CD34, CD14, CD19, CD11b, and HLA-DR.

14. The method of claim 1, wherein the MSC-conditioned medium is obtained from a cell culture comprising MSCs that are positive for expression of CD105 and are negative for expression of CD34.

15. A method for treating, preventing, or reducing the risk of ischemia reperfusion injury in a transplant tissue comprising:
a. administering a therapeutically effective amount of an MSC (mesenchymal stem cell)-conditioned medium to a transplant donor prior to excision of a transplant tissue from the transplant donor; and
b. excising the transplant tissue from the transplant donor.

16-26. (canceled)

27. A method for treating, preventing, or reducing the risk of ischemia reperfusion injury in a transplant tissue comprising:
a. administering a therapeutically effective amount of an MSC (mesenchymal stem cell)-conditioned medium to a transplant donor prior to excision of a transplant tissue from the transplant donor;
b. excising the transplant tissue from the transplant donor; and
c. transplanting the excised transplant tissue into a transplant recipient.

28-48. (canceled)
